(19) United States
(12) Patent Application Publication
(10) Pub. No.: US 2019/0142953 A1
(43) Pub. Date: May 16, 2019

(54) Dendrimer-Drug Conjugates, Hydrogel Compositions, and Methods

(71) Applicant: Massachusetts Institute of Technology, Cambridge, MA (US)

(72) Inventors: Elazer R. Edelman, Brookline, MA (US); Natalie Artzi, Brookline, MA (US); Nuria Oliva, Somerville, MA (US)

(21) Appl. No.: 16/300,161

(22) PCT Filed: May 10, 2017
(86) PCT No.: PCT/US17/31968

§ 371 Date: Nov. 9, 2018

Related U.S. Application Data
(60) Provisional application No. 62/333,893, filed on May 10, 2016.

(51) Int. Cl. A61K 47/59; A61K 47/62; A61K 47/69; A61K 47/60; A61P 35/00; A61K 31/704; A61M 5/19

(52) U.S. Cl. CPC A61K 47/59; A61K 47/62; A61K 47/6903; A61M 5/19; A61P 35/00; A61K 31/704; A61K 47/60

(57) ABSTRACT

Abstract of the Disclosure Provided herein are compositions that include a dendrimer to which a drug and a binding peptide are conjugated. The binding peptide may be configured to bind to a receptor that is overexpressed by a diseased cell, such as a cancer cell. The compositions may include a hydrogel in which the dendrimer-drug conjugate is dispersed. Methods of drug delivery and kits also are provided.

### CROSS-REFERENCE TO RELATED APPLICATIONS

This application claims priority to U.S. Provisional Patent Application No. 62/333,893, filed May 10, 2016, which is incorporated herein by reference.

### BACKGROUND

Chemotherapy is an effective cancer treatment modality; however, the side effects of chemotherapy can be devastating for patients due to its toxicity to non-cancerous cells. Systemic delivery of chemotherapeutic drugs also can result in low drug accumulation at a target tumor site, and/or high drug retention in the liver and kidneys.

Tissues and cells are transient entities that may be capable of altering their surroundings in response to one or more pathologies. Tissues and cells, for example, may alter protein and/or enzyme concentrations, pH, redox potential, oxygenation, inflammation, and tumor immunity. These pathophysiological variations typically manifest in cancer, wherein a complex tumor microenvironment may exhibit several cell types, tissue chemistry, and morphology, along with mechanical stresses. Disease-specific stimuli, such as pH, proteases (MMPs), phospholipases (sPLA₂), or nucleic acids, among others, have been exploited in an attempt to allow triggered and/or targeted drug release.

Biomaterials have been used as vehicles for intravenous delivery of chemotherapeutic agents in an effort to improve cargo protection, avoid rapid clearance, and increase circulation time. Although systemic targeting may enable tumor accumulation due to the enhanced permeability and retention (EPR) effect, current biomaterial design has focused on enhancing vehicle accumulation at the tumor site by adding moieties to actively target tumor cells. However, these targeting moieties typically are not selective to cancer cells only, and may affect healthy cells in surrounding tissues, which may elicit undesired toxicity. Moreover, cargo bioavailability typically is no more than marginally improved.

Dendrimers have been used as vehicles for drug delivery and genetic material delivery, due at least in part to the ability of dendrimers to enter cells and escape the endosome. However, dendrimers are typically associated with high levels of toxicity and/or the inability to discern healthy cells and tumor cells. Efforts to address these disadvantages have included derivatizing dendrimers with molecules that provide specific cancer cell targeting, such as folic acid. None of these derivatized dendrimers, however, has been shown to be selectively uptaken by cancer cells.

There remains a need for compositions that are capable of delivering a drug selectively and/or locally to diseased cells, including compositions that are stable and/or can deliver a drug in a sustained manner.

### BRIEF SUMMARY

Improved compositions, including dendritic pro-drug compositions, are provided that can selectively and/or locally deliver a drug to diseased cells, including cancer cells. The compositions may include a hydrogel, which may facilitate local and/or sustained delivery of the drug.

In one aspect, compositions are provided herein that include a first dendrimer having at least two branches with one or more surface groups; a drug conjugated to the first dendrimer; and a binding peptide conjugated to the first dendrimer; wherein the binding peptide is configured to bind to a receptor overexpressed by a diseased cell. In embodiments, the compositions include a hydrogel in which the first dendrimer is dispersed.

In another aspect, methods of drug delivery are provided. The methods, in embodiments, include providing a first solution that includes a first polymer component comprising a first polymer having one or more aldehydes; providing a second solution that includes at least one of (i) a second dendrimer comprising at least two branches with one or more surface groups, wherein about 25% to 100% of the surface groups have at least one primary or secondary amine, and (ii) a second polymer component that includes a second polymer having one or more amines; combining the first and second solutions together to produce a hydrogel composite; and contacting one or more biological tissues with the hydrogel composite, wherein at least one of the first solution and the second solution includes a first dendrimer to which a drug and a binding peptide have been conjugated.

In yet another aspect, kits for making a hydrogel composite are provided. The kit may include a first part that includes a first solution, and a second part that includes a second solution. The first solution may include a first polymer component that includes a first polymer having one or more aldehydes. The second solution may include at least one of (i) a second dendrimer having at least two branches with one or more surface groups, wherein about 25% to 100% of the surface groups include at least one primary or secondary amine, and (ii) a second polymer component including a second polymer having one or more amines. At least one of the first solution and the second solution includes a first dendrimer to which a drug and a binding peptide have been conjugated.

In a further aspect, methods for local delivery of a drug to a biological tissue are provided. The methods may include applying to the biological tissue a hydrogel in which a dendrimer has been dispersed, and permitting the dendrimer to diffuse from the composition into the biological tissue. A drug and a binding peptide may be conjugated to the dendrimer.

### BRIEF DESCRIPTION OF THE DRAWINGS

FIG. 1 depicts one embodiment of a kit containing the components of a hydrogel composite or composition.

FIG. 2A depicts the dose response of epidermal growth factor (EGFR)-overexpressing cells to one embodiment of a dendritic pro-drug, one embodiment of a dendrimer-peptide without conjugated drug, and free doxorubicin.

FIG. 2B depicts the dose response of healthy mammary epithelial cells to one embodiment of a dendritic pro-drug, one embodiment of a dendrimer-peptide without conjugated drug, and free doxorubicin.

FIG. 2C depicts the cancer-specific cytocidal effect at a concentration of 10 μM of one embodiment of a dendritic pro-drug compared to equivalent concentrations of one embodiment of a dendrimer-peptide without conjugated drug, and free doxorubicin.

FIG. 3A depicts the bioluminescence fold increase of tumors treated with single or multiple dose regimens of one embodiment of a dendritic pro-drug.

FIG. 3B depicts the bioluminescence fold increase of tumors treated locally or systemically with one embodiment of a dendritic pro-drug.

FIG. 3C depicts the bioluminescence fold increase of tumors that are untreated or treated locally with an embodiment of a dendritic pro-drug.

### DETAILED DESCRIPTION

Improved compositions are provided, including dendritic pro-drugs, that can selectively and/or locally deliver a drug to diseased cells, including cancer cells. In embodiments, the compositions can have a cytotoxic effect on cancer cells, while having little or no effect on adjacent healthy cells in vitro and in vivo. The compositions may include a hydrogel, which may permit local and/or sustained delivery of drug, thereby providing, in some embodiments, longer therapeutic times, higher tumor effective doses, or a combination thereof.

Generally, in embodiments, the compositions provided herein may be used on or in any diseased cells and/or biological tissue that may include or may be associated with diseased cells. The diseased cells may include cancer cells. For example, the compositions may be used on or in any internal or external biological tissues, lumens, orifices, or cavities. The biological tissues may be those of a human or other mammal.

In some embodiments, the compositions include a hydrogel, and the hydrogel may serve as a matrix material for controlled delivery of a dendritic pro-drug. The compositions can be applied to a tissue site in a human or other animal patient, for example, during a surgical or other medical procedure. For example, the compositions may be applied to a tissue bed following resection of a tumor.

### Dendritic Pro-Drugs and Hydrogel Composites

Generally, the compositions provided herein may include a first dendrimer having at least two branches with one or more surface groups; a drug conjugated to the first dendrimer; and a binding peptide conjugated to the first dendrimer. The first dendrimer to which a drug is conjugated may be referred to herein as a dendritic “pro-drug,” whether or not the drug needs to be released from the conjugate to exert its therapeutic or prophylactic effects.

The compositions provided herein may include one or more first dendrimer molecules to which a drug and a binding peptide have been conjugated. Generally, the number of drug molecules conjugated to each first dendrimer molecule, and the number of binding peptide molecules conjugated to each first dendrimer molecule is limited only by the size of each first dendrimer molecule and/or the number of atoms in each first dendrimer molecule to which a drug or binding peptide may be conjugated. The number of drug molecules, the number of binding peptide molecules, the ratio of drug molecules to binding peptide molecules, or a combination thereof, may be adjusted to modify one or more properties of a composition, such as the potency of the composition, the affinity of the composition for one or more receptors, or a combination thereof.

In embodiments, an average of about 10 to about 80 molecules of a binding peptide are conjugated to each molecule of the first dendrimer. In some embodiments, an average of about 20 to about 70 molecules of a binding peptide are conjugated to each molecule of the first dendrimer. In further embodiments, an average of about 30 to about 60 molecules of a binding peptide are conjugated to each molecule of the first dendrimer. In additional embodiments, an average of about 30 to about 50 molecules of a binding peptide are conjugated to each molecule of the first dendrimer.

In embodiments, an average of about 4 to about 20 molecules of a drug are conjugated to each molecule of the first dendrimer. In some embodiments, an average of about 4 to about 16 molecules of a drug are conjugated to each molecule of the first dendrimer. In further embodiments, an average of about 4 to about 12 molecules of a drug are conjugated to each molecule of the first dendrimer. In embodiments, an average of about 6 to about 10 molecules of a drug are conjugated to each molecule of the first dendrimer.

In embodiments, an average of about 10 to about 80 molecules, about 20 to about 70 molecules, about 30 to about 60 molecules, or about 30 to about 50 molecules of a binding peptide are conjugated to each molecule of the first dendrimer; and an average of about 4 to about 20 molecules of a drug are conjugated to each molecule of the first dendrimer. In some embodiments, an average of about 10 to about 80 molecules, about 20 to about 70 molecules, about 30 to about 60 molecules, or about 30 to about 50 molecules of a binding peptide are conjugated to each molecule of the first dendrimer; and an average of about 4 to about 16 molecules of a drug are conjugated to each molecule of the first dendrimer. In further embodiments, an average of about 10 to about 80 molecules, about 20 to about 70 molecules, about 30 to about 60 molecules, or about 30 to about 50 molecules of a binding peptide are conjugated to each molecule of the first dendrimer; and an average of about 4 to about 12 molecules of a drug are conjugated to each molecule of the first dendrimer. In additional embodiments, an average of about 10 to about 80 molecules, about 20 to about 70 molecules, about 30 to about 60 molecules, or about 30 to about 50 molecules of a binding peptide are conjugated to each molecule of the first dendrimer; and an average of about 6 to about 10 molecules of a drug are conjugated to each molecule of the first dendrimer.

The average number of drug and/or binding peptide molecules conjugated to each dendrimer molecule may be determined according to the procedures of Example 1.

A first molecule, such as a drug molecule and/or a binding peptide molecule, is “conjugated” to a second molecule, such as a first dendrimer, when an atom of the first molecule is [1] covalently bonded to an atom of the second molecule, [2] covalently bonded to a first atom of a linker, wherein the linker includes a second atom covalently bonded to the second molecule, or [3] a combination thereof. Non-limiting examples of linkers include the drug linkers and binding peptide linkers provided herein.

In embodiments, the compositions described herein include a drug linker. The drug linker may be covalently bonded to [1] an atom of a first dendrimer, such as an atom of one or more surface groups, and [2] an atom of a drug. The drug linker generally may include one or more biocompatible molecules that are capable of covalently bonding to a first dendrimer, a drug, each other, or a combination thereof. The structure and/or size of the drug linker may be selected to adjust one or more properties of the compositions, such as drug accessibility. In one embodiment, the drug linker includes a cysteine linker, an aconityl linker, a PEGylated amine-to-sulfhydryl crosslinker, or a combination thereof.

The PEGylated amine-to-sulfhydryl crosslinker may be any molecule that includes [1] a PEG moiety of one or more monomers (e.g., 1 to 1,000 monomers), [2] at least one moiety capable of forming a covalent bond with an amine, and [3] at least one moiety capable of forming a covalent bond with a thiol. For example, the PEGylated amine-to-sulfhydryl crosslinker may include a 2,5-dioxopyrrolidin-1-yl acetate moiety, which is capable of forming a covalent bond with an amine, and a 1H-pyrrole-2,5-dione moiety, which is capable of forming a covalent bond with a thiol, wherein the two moieties are connected by a moiety that includes a PEG of one or more monomers (e.g., 1 to 1,000 monomers), one or more other functional groups (such as an amide), or a combination thereof.

In one embodiment, the drug linker is or includes a PEGylated amine-to-sulfhydryl crosslinker having the following structure, which is referred to herein as SM(PEG)₂:

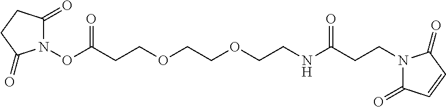 [SMILES: O=C(CCN1C(=O)C=CC1=O)NCCOCCOCCC(=O)ON1C(=O)CCC1=O]

In one embodiment, the drug linker is or includes aconityl, which has the following structure:

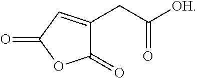 [SMILES: O=C(O)CC1=CC(=O)OC1=O]

In one embodiment, a drug, such as doxorubicin (DOX), is conjugated to a dendrimer, such as a PAMAM dendimer, through an aconityl linker.

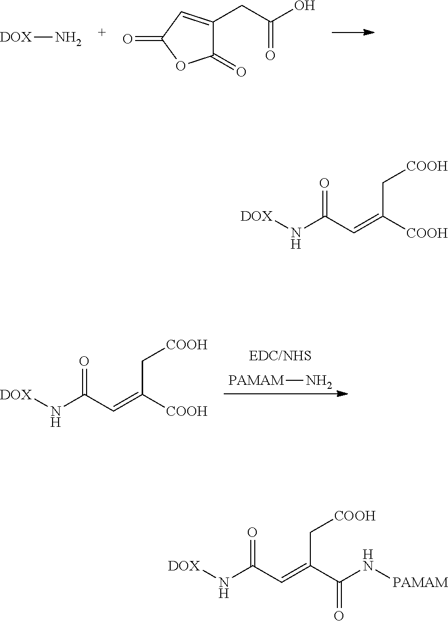

In one embodiment, the drug linker includes SM(PEG)₂, cysteine and aconityl, which may covalently bond to each other as shown in the following structure:

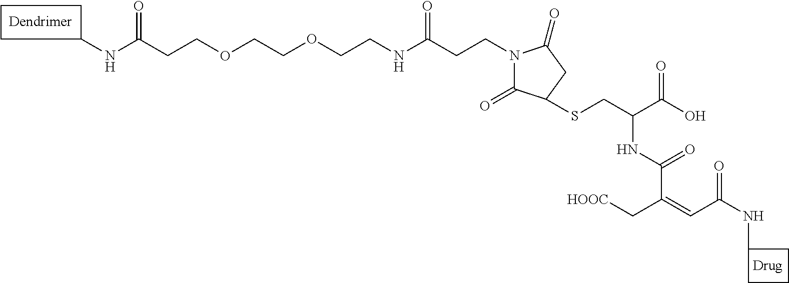 [SMILES: O=C(O)C/C(=C/C(=O)NC1CCC1)C(=O)NC(CSC1CC(=O)N(CCC(=O)NCCOCCOCCC(=O)NC2CCC2)C1=O)C(=O)O]

In embodiments, the compositions described herein include a binding peptide linker. The binding peptide linker may be covalently bonded to [1] an atom of a first dendrimer, such as an atom of one or more surface groups, and [2] an atom of a binding peptide. The binding peptide linker generally may include one or more biocompatible molecules that are capable of covalently bonding to a first dendrimer and a binding peptide. The structure and/or size of the binding peptide linker may be selected to adjust one or more properties of the compositions, such as the affinity of the composition for a receptor. In one embodiment, the binding peptide linker is a PEGylated amine-to-sulfhydryl crosslinker, such as SM(PEG)₂.

The SM(PEG)₂ binding peptide linker may covalently bond with an amine of a surface group of a dendrimer, and a thiol of a binding peptide, thereby producing the following structure:

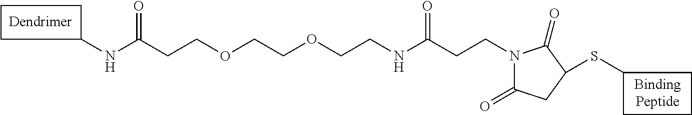 [SMILES: O=C(CCN1C(=O)CC(SC2CCC2)C1=O)NCCOCCOCCC(=O)NC1CCC1]

A binding peptide may be modified to accommodate covalently bonding the binding peptide to a binding peptide linker, such as a PEGylated amine-to-sulfhydryl crosslinker. For example, a binding peptide may be modified with one or more molecules that include a thiol functional group, such as cysteine. The thiol functional group may react with a 1H-pyrrole-2,5-dione moiety of a PEGylated amine-to-sulfhydryl crosslinker to form a covalent bond. Other molecules, such as one or more amino acids, also may be used to modify a binding peptide.

In embodiments, the compositions described herein include a drug linker and a binding peptide linker. In one embodiment, [1] the drug linker includes an aconityl linker, a cysteine linker, SM(PEG)₂, or a combination thereof, and [2] the binding peptide linker is a PEGylated amine-to-sulfhydryl crosslinker, such as SM(PEG)₂.

The first dendrimer may be substituted with one or more functional groups, such as amines, that are capable of reacting with the one or more functional groups of a component of a hydrogel, as described herein, a drug, a binding peptide, a drug linker, a binding peptide linker, or a combination thereof.

In some embodiments, the first dendrimer has amines on at least a portion of its surface groups, which are commonly referred to as “terminal groups” or “end groups.” These amines can be capable of reacting with the one or more functional groups of a component of a hydrogel, as described herein, a drug, a binding peptide, a drug linker, a binding peptide linker, or a combination thereof. The first dendrimer may have amines on about 5% to 100%, about 10% to 100%, or about 25% to 100% of its surface groups. In some embodiments, the first dendrimer has amines on 100% of its surface groups. In one embodiment, the first dendrimer has amines on less than 75% of its surface groups. As used herein, the term “first dendrimer” refers to any compound that has a polyvalent core covalently bonded to two or more dendritic branches, and is functionalized with at least one of a drug and a binding peptide. In some embodiments, the polyvalent core is covalently bonded to three or more dendritic branches. In one embodiment, the amines are primary amines. In another embodiment, the amines are secondary amines. In yet another embodiment, one or more surface groups have at least one primary and at least one secondary amine.

In one embodiment, the first dendrimer extends through at least 2 generations. In another embodiment, the first dendrimer extends through at least 3 generations. In yet another embodiment, the first dendrimer extends through at least 4 generations. In still another embodiment, the first dendrimer extends through at least 5 generations. In a further embodiment, the first dendrimer extends through at least 6 generations. In still a further embodiment, the first dendrimer extends through at least 7 generations.

In one embodiment, the first dendrimer has a molecular weight of about 1,000 to about 1,000,000 Daltons. In a further embodiment, the first dendrimer has a molecular weight of about 3,000 to about 120,000 Daltons. In another embodiment, the first dendrimer has a molecular weight of about 10,000 to about 100,000 Daltons. In yet another embodiment, the first dendrimer has a molecular weight of about 20,000 to about 40,000 Daltons. Unless specified otherwise, the “molecular weight” of the first dendrimer refers to the number average molecular weight.

Generally, the first dendrimer may be made using any known methods. In one embodiment, the first dendrimer is made by oxidizing a starting first dendrimer having surface groups comprising at least one hydroxyl group so that at least a portion of the surface groups comprise at least one amine. In another embodiment, the first dendrimer is made by oxidizing a starting generation 5 (G5) first dendrimer having surface groups comprising at least one hydroxyl group so that at least a portion of the surface groups comprise at least one amine. In yet another embodiment, the first dendrimer is made by oxidizing a starting G5 first dendrimer having surface groups comprising at least one hydroxyl group so that about 5% to 100%, about 10% to 100%, or about 25% to 100% of its surface groups comprise at least one amine. In a particular embodiment, the first dendrimer is a G5 first dendrimer having primary amines on about 5% to 100%, about 10% to 100%, or about 25% to 100% of its surface groups. In a certain embodiment, the first dendrimer is a G5 first dendrimer having primary amines on about 25% of the first dendrimer's surface groups.

In one embodiment, the first dendrimer is a poly(amidoamine)-derived (PAMAM) first dendrimer. In another embodiment, the first dendrimer is a G5 PAMAM-derived first dendrimer. In yet another embodiment, the first dendrimer is a G5 PAMAM-derived first dendrimer having primary amines on about 5% to 100%, about 10% to 100%, or about 25% to 100% of its surface groups. In a further embodiment, the first dendrimer is a G5 PAMAM-derived first dendrimer having primary amines on about 25% of the first dendrimer's surface groups.

In one embodiment, the first dendrimer is a poly(propyleneimine)-derived first dendrimer.

In embodiments, the compositions provided herein include a hydrogel. The first dendrimer may be dispersed in the hydrogel. The first dendrimer may be dispersed at least substantially evenly in the hydrogel, or unevenly in the hydrogel. The concentration of the first dendrimer in the hydrogel may be about 5 μM to about 75 μM, about 15 μM to about 65 μM, or about 25 μM to about 50 μM.

Generally, the compositions described herein may include any biocompatible hydrogel. In such embodiments, the hydrogel may serve as a matrix material for controlled delivery of drug, localized drug delivery, or a combination thereof. Methods of locally delivering a drug may include applying to a biological tissue, such as a human tissue, a drug delivery composition as provided herein, and permitting the at least one drug conjugated to the first dendrimer to diffuse from the composition into the biological tissue. The hydrogel may adhere to one or more biological tissues, thereby reducing or eliminating the risk of unwanted material migration following application of the composition to one or more selected tissue sites. The hydrogel generally may be degradable, injectable, or a combination thereof.

The hydrogel may include a contact product of [1] a first solution that includes the first polymer component described herein, and [2] a second solution that includes the second polymer component and/or the dendrimer component described herein. The first dendrimer may be added to the hydrogel after hydrogel formation; the first dendrimer may be added to the first solution, the second solution, or both the first and second solution prior to hydrogel formation; or a combination thereof. The first dendrimer may be present in the first solution, the second solution, or a combination thereof in an amount sufficient to impart the resulting hydrogel with a concentration of the first dendrimer of about 5 μM to about 75 μM, about 15 μM to about 65 μM, or about 25 μM to about 50 μM. The first dendrimer may be disposed in the solution having components with which the first dendrimer is incapable of reacting. For example, if [1] the first solution includes a component that includes aldehydes, and [2] the first dendrimer includes amines and at least a portion of those amines are not covalently bonded to a linker, a drug, or a binding peptide, then the first dendrimer may be disposed in the second solution to avoid the possibility of the first dendrimer reacting with the aldehydes of the first solution prior to hydrogel formation. If, however, a reaction between the first dendrimer and a component of the hydrogel may be beneficial prior to hydrogel formation, then the first dendrimer may be disposed in the first solution.

The rate of drug delivery may be controlled, at least in part, by imparting the first dendrimer with one or more functional groups capable of reacting with a functional group of at least one component of the hydrogel in which the first dendrimer is dispersed. If the first dendrimer does not include functional groups capable of reacting with a functional group of at least one component of the hydrogel in which the first dendrimer is dispersed, then the rate of drug delivery may be dictated by the diffusion of the first dendrimer from the hydrogel. If the first dendrimer does include a functional group capable of reacting with a functional group of at least one component of the hydrogel, then the rate of drug delivery may be dictated by the degradation rate of the hydrogel, the diffusion of the first dendrimer from the hydrogel, or a combination thereof.

Generally, the hydrogel composites and compositions, including drug delivery compositions, provided herein may be formed by combining a first solution and a second solution as described herein. The first solution and the second solution may be aqueous macromer solutions. The first solution and/or the second solution may independently include water, phosphate buffer saline (PBS), Dulbecco's Modified Eagle's Medium (DMEM), or any combination thereof.

The first solution, in embodiments, includes the first dendrimer and a first polymer component. The first solution, in other embodiments, includes a first polymer component without a first dendrimer.

The second solution may include at least one of a second dendrimer and a second polymer component. The second dendrimer and/or second polymer component generally have one or more functional groups capable of reacting with the one or more functional groups on the first polymer. The second dendrimer and/or second polymer component, in particular embodiments, include one or more amines. The second solution, in other embodiments, also includes the first dendrimer.

The first solution and the second solution, in embodiments, are combined to form the hydrogel composites and compositions described herein. When combined, the aldehyde groups of the first solution may react with the amines that are present in the second solution.

This reaction is referred to herein as “curing” or “gelling.”

In embodiments, the first dendrimer is present in the first solution. In some embodiments, the first dendrimer is present in the first solution and the second solution. In further embodiments, the first dendrimer is present in the second solution. When the first solution and the second solution include the first dendrimer, the first dendrimer of the first solution and the second solution may have the same or different compositions. For example, the first solution may include a first dendrimer conjugated to a first drug and/or first binding peptide, and the second solution may include a first dendrimer conjugated to a second drug and/or second binding peptide. As a further example, the first solution may include a first dendrimer lacking functional groups capable of reacting with the components of the first solution, and the second solution may include a first dendrimer lacking functional groups capable of reacting with the components of the second solution.

In embodiments, the first dendrimer is substantially evenly dispersed in the first solution. In other embodiments, the first dendrimer is substantially evenly dispersed in the first solution and the second solution. In further embodiments, the first dendrimer is evenly dispersed in the second solution. Although the first dendrimer is evenly dispersed in preferred embodiments, other embodiments may not have an even dispersement of the first dendrimer.

In embodiments, the concentration of the first dendrimer in the first solution is about 0.01% to about 30% by weight of the first solution. In some embodiments, the concentration of the first dendrimer in the first solution is about 0.01% to about 25% by weight of the first solution. In further embodiments, the concentration of the first dendrimer in the first solution is about 0.01% to about 20% by weight of the first solution. In still further embodiments, the concentration of the first dendrimer in the first solution is about 0.01% to about 15% by weight of the first solution.

In embodiments, the concentration of the first dendrimer in the second solution is about 0.01% to about 30% by weight of the second solution. In some embodiments, the concentration of the first dendrimer in the second solution is about 0.01% to about 25% by weight of the second solution. In further embodiments, the concentration of the first dendrimer in the second solution is about 0.01% to about 20% by weight of the second solution. In still further embodiments, the concentration of the first dendrimer in the second solution is about 0.01% to about 15% by weight of the second solution.

In embodiments, the concentration of the first dendrimer in the hydrogel composites or compositions described herein is about 0.01% to about 10% by weight of the hydrogel composite or composition. In some embodiments, the concentration of the first dendrimer in the hydrogel composites or compositions described herein is about 0.01% to about 8% by weight of the hydrogel composite or composition. In certain embodiments, the concentration of the first dendrimer in the hydrogel composites or compositions described herein is about 0.01% to about 6% by weight of the hydrogel composite or composition. In particular embodiments, the concentration of the first dendrimer in the hydrogel composites or compositions described herein is about 0.01% to about 5% by weight of the hydrogel composite or composition.

In embodiments, the concentration of first polymer component in the first solution is about 0.01% to about 40% by weight of the first solution. In further embodiments, the concentration of first polymer component in the first solution is about 0.01% to about 30% by weight of the first solution. In some embodiments, the concentration of first polymer component in the first solution is about 0.01% to about 20% by weight of the first solution. In a particular embodiment, the concentration of first polymer component in the first solution is about 20% by weight of the first solution. In additional embodiments, the concentration of first polymer component in the first solution is about 0.01% to about 10% by weight of the first solution. Typically, the concentration may be tailored and/or adjusted based on the particular application, tissue type, and/or the type and concentration of dendrimer and/or second polymer component used.

In embodiments, the concentration of the first polymer component in the hydrogel composites or compositions described herein is about 0.01% to about 20% by weight of the hydrogel composite or composition. In further embodiments, the concentration of the first polymer component in the hydrogel composites or compositions described herein is about 0.01% to about 15% by weight of the hydrogel composite or composition. In some embodiments, the concentration of the first polymer component in the hydrogel composites or compositions described herein is about 0.01% to about 10% by weight of the hydrogel composite or composition. In still further embodiments, the concentration of the first polymer component in the hydrogel composites or compositions described herein is about 0.01% to about 7% by weight of the hydrogel composite or composition.

In embodiments, the total concentration of second dendrimer and second polymer component in the second solution is about 0.01% to about 40% by weight of the second solution. In further embodiments, the total concentration of second dendrimer and second polymer component in the second solution is about 0.01% to about 30% by weight of the second solution. In some embodiments, the total concentration of second dendrimer and second polymer component in the second solution is about 0.01% to about 20% by weight of the second solution. In additional embodiments, the total concentration of second dendrimer and second polymer component in the second solution is about 0.01% to about 10% by weight of the second solution. In a particular embodiment, the total concentration of second dendrimer and second polymer component in the second solution is about 25% by weight of the second solution. Typically, the concentration may be tailored and/or adjusted based on the particular application, tissue type, and/or the type and concentration of first polymer component used. As used herein, the phrase “total concentration of second dendrimer and second polymer component” refers to the sum of the concentration of dendrimer and the concentration of the second polymer component. The phrase does not imply that both a second dendrimer and a second polymer component must be present in the second solution. The second solution may include a second dendrimer, second polymer component, or both a second dendrimer and second polymer component.

In embodiments, the total concentration of second dendrimer and second polymer component in the hydrogel composites or compositions described herein is about 0.01% to about 20% by weight of the hydrogel composite or composition. In further embodiments, the total concentration of second dendrimer and second polymer component in the hydrogel composites or compositions described herein is about 0.01% to about 15% by weight of the hydrogel composite or composition. In some embodiments, the total concentration of second dendrimer and second polymer component in the hydrogel composites or compositions described herein is about 0.01% to about 10% by weight of the hydrogel composite or composition. In still further embodiments, the total concentration of second dendrimer and second polymer component in the hydrogel composites or compositions described herein is about 0.01% to about 7% by weight of the hydrogel composite or composition.

### Binding Peptides

The first dendrimer may be conjugated to a binding peptide that is configured to bind to a receptor overexpressed by a diseased cell, such as a cancer cell. The binding peptide, therefore, may result in the selective cellular uptake of the compositions provided herein in the target diseased cells. The binding peptides, upon cellular uptake, also may result in receptor-mediated endocytosis (RME). The binding peptides used in the compositions provided herein may generally include any binding peptide that is configured to bind to one or more receptors of diseased cells, such as receptors that are upregulated by diseased cells.

For example, epidermal growth factor receptor (EGFR) expression is upregulated in multiple cancer types, such as breast, head, neck, cervical, ovarian, bladder, esophageal, endometrial, lung, and colorectal cancer. The upregulation of EGFR may be correlated with increased recurrence-rate and/or reduced overall survival. Therefore, in one embodiment, the binding peptide of the compositions provided herein is an EGF mimicking peptide (EGFmp) that is capable of binding to EGFR.

Since other binding peptides may be used, the compositions herein may be used to deliver a drug to many types of cancer cells by conjugation of other binding peptides, such as growth factor mimicking peptides, to target other commonly overexpressed receptors in cancer cells, such as FGFR-2, VEGFR, and PDGFR.

The binding peptide may be selected from an EGF mimicking peptide, an FGF-2 mimicking peptide, a VEGF mimicking peptide, a PDGF mimicking peptide, or a combination thereof. These binding peptides are configured to bind, respectively, to the following receptors: EGFR, FGFR-2, VEGFR, and PDGFR.

The binding peptide may include one type of binding peptide, or two or more types of binding peptide. Each type of binding peptide may be configured to bind to a different receptor.

The binding peptides may be synthetic peptides. Not wishing to be bound by any particular theory, it is believed that synthetic peptides may result in higher stability when the composition is in solution, and/or higher and more selective uptake. It also is believed that synthetic peptides may interact with growth factor receptors, and possibly elicit RME without activating the downstream signaling pathway.

### Drugs

Generally, any drug may be conjugated to a first dendrimer. The drug that is conjugated to a first dendrimer may include a single type of drug or two or more types of drug. Since the response to drugs can vary from patient to patient, the compositions provided herein may be personalized on a patient-by-patient basis. In embodiments, the drug includes one or more chemotherapeutic agents, one or more anti-angiogenic agents, or a combination thereof.

In embodiments, the drug conjugated to a first dendrimer includes one or more chemotherapeutic agents. A “chemotherapeutic agent” is a chemical compound useful in the treatment of cancer. Examples of chemotherapeutic agents include, but are not limited to, alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In one embodiment, the one or more chemotherapeutic agents includes an anthracycline drug. In a particular embodiment, the one or more chemotherapeutic agents includes doxorubicin.

### First Polymer Component

The first polymer component generally includes a first polymer with one or more functional groups capable of reacting with one or more functional groups on a biological tissue and/or one or more functional groups on the dendrimer component and/or second polymer component of the second solution. The one or more functional groups of the first polymer component also may be capable of reacting with one or more functional groups on the first dendrimer, such as the one or more surface groups of the first dendrimer. The first polymer component, in embodiments, comprises a first polymer having one or more aldehyde groups.

The polymers of the first polymer component may be selected from any biocompatible polymers capable of forming or imparting certain characteristics to the hydrogel composites and compositions described herein. The polymers of the first polymer component, for example, may be selected from at least one polysaccharide, at least one hydrophilic polymer, at least one hydrophobic polymer, or combinations thereof.

In one embodiment, the first polymer component includes a first polymer that is a polysaccharide having one or more aldehyde groups. In a certain embodiment, the first polymer component includes a first polymer that is a hydrophilic polymer having one or more aldehyde groups. In another embodiment, the first polymer component includes a first polymer that is a polysaccharide having one or more aldehyde groups, and a hydrophilic polymer. In further embodiments, the first polymer component includes a first polymer that is a polysaccharide having one or more aldehyde groups, a hydrophilic polymer, and a hydrophobic polymer. In some embodiments, the first polymer component comprises a first polymer that includes a polysaccharide and a hydrophilic polymer, wherein both the polysaccharide and hydrophilic polymer have one or more aldehyde groups. Therefore, as used herein, the phrase “first polymer” refers to the one or more polymers of the first polymer component that include one or more functional groups, e.g., aldehydes, that are capable of reacting with a biological tissue and/or the functional groups of the dendrimer component and/or second polymer component. In still further embodiments, the first polymer component comprises a first polymer that includes a polysaccharide and a hydrophilic polymer, wherein both the polysaccharide and hydrophilic polymer have one or more aldehyde groups, and a hydrophobic polymer.

In embodiments, the first polymer comprises at least one polysaccharide. The at least one polysaccharide may be linear, branched, or have both linear and branched sections within its structure. The at least one polysaccharide may be anionic, cationic, nonionic, or a combination thereof. Generally, the at least one polysaccharide may be natural, synthetic, or modified—for example, by crosslinking, altering the polysaccharide's substituents, or both. In one embodiment, the at least one polysaccharide is plant-based. In another embodiment, the at least one polysaccharide is animal-based. In yet another embodiment, the at least one polysaccharide is a combination of plant-based and animal-based polysaccharides. Non-limiting examples of polysaccharides include, but are not limited to, dextran, dextrin, chitin, starch, agar, cellulose, hyaluronic acid, derivatives thereof, such as cellulose derivatives, or a combination thereof.

In embodiments, the at least one polysaccharide is nonionic. Non-limiting examples of nonionic polysaccharides include dextran, dextrin, and cellulose derivatives. In other embodiments, the at least one polysaccharide is anionic. Non-limiting examples of anionic polysaccharides include hyaluronic acid, chondroitin sulfate, alginate, and cellulose gum. In further embodiments, the at least one polysaccharide is cationic. The cationic character may be imparted by substituting the at least one polysaccharide with positively charge groups, such as trimethylammonium groups. Non-limiting examples of cationic polysaccharides include chitosan, cationic guar gum, cationic hydroxyethylcellulose, or other polysaccharides modified with trimethylammonium groups to confer positive charge.

In embodiments, the first polymer component comprises one or more hydrophilic polymers. The hydrophilic polymers are modified, in some embodiments, to confer degradability. For example, the hydrophilic polymers may be modified with polyester groups in order to impart degradability of the hydrophilic polymer. In particular embodiments, the hydrophilic polymers are substituted with one or more functional groups, such as aldehydes, that are capable of reacting with biological tissue and/or the functional groups of the dendrimer and/or second polymer component, such as amines. Generally, any biocompatible hydrophilic polymer may be used. Non-limiting examples of hydrophilic polymers include poly(vinyl alcohol), poly(acrylic acid), poly(acrylamide), poly(ethylene oxide), or combinations thereof.

In embodiments, the first polymer component comprises one or more hydrophobic polymers. The hydrophobic polymers may be modified with pendant hydrophilic polymers to adjust their characteristics. Non-limiting examples of hydrophobic polymers include polycaprolactam, poly(lactic acid), polycaprolactone, or combinations thereof.

In certain embodiments, the first polymer has a molecular weight of about 1,000 to about 1,000,000 Daltons. In one embodiment, the first polymer has a molecular weight of about 5,000 to about 15,000 Daltons. Unless specified otherwise, the “molecular weight” of the polymer refers to the number average molecular weight. The molecular weight may be adjusted to attain certain properties, as known to those of skill in the art.

Generally, the one or more functional groups of the first polymer may be present in a number sufficient to form the hydrogel composites and compositions described herein. In certain embodiments, the first polymer's degree of functionalization is adjustable. The “degree of functionalization” generally refers to the number or percentage of groups on the polymer that are replaced or converted to the desired one or more functional groups. The one or more functional groups, in particular embodiments, include aldehydes. In one embodiment, the degree of functionalization is adjusted based on the type of tissue to which the hydrogel composites or compositions is applied, the concentration(s) of the various components, and/or the type of polymer(s) or dendrimer(s) used in the first and second solutions. In one embodiment, the degree of functionalization is about 10% to about 75%. In another embodiment, the degree of functionalization is about 25% to about 60%. In yet another embodiment, the degree of functionalization is about 40% to about 50%.

In one embodiment, the first polymer is a polysaccharide having about 10% to about 75% of its vicinal hydroxyl groups converted to aldehydes. In another embodiment, the first polymer is a polysaccharide having about 25% to about 75% of its vicinal hydroxyl groups converted to aldehydes.

In one embodiment, the first polymer is dextran with a molecular weight of about 10 kDa. In another embodiment, the first polymer is dextran having about 50% of its vicinal hydroxyl group converted to aldehydes. In a further embodiment, the first polymer is dextran with a molecular weight of about 10 kDa and about 50% of its vicinal hydroxyl groups converted to aldehydes.

In some embodiments, a polysaccharide and/or hydrophilic polymer is oxidized to include a desired percentage of one or more aldehyde functional groups. Generally, this oxidation may be conducted using any known means. For example, suitable oxidizing agents include, but are not limited to, periodates, hypochlorites, ozone, peroxides, hydroperoxides, persulfates, and percarbonates. In one embodiment, the oxidation is performed using sodium periodate. Typically, different amounts of oxidizing agents may be used to alter the degree of functionalization. In addition to, or independently of, other methods, aldehyde groups can be grafted onto the polymer backbone using known bioconjugation techniques in the event that oxidative methods are unsuitable.

### Second Polymer Component

The second polymer component generally includes a second polymer with one or more functional groups capable of reacting with one or more functional groups of the first polymer of the first polymer component. The second polymer component, in embodiments, comprises a second polymer having one or more amines. The amines may be primary amines, secondary amines, or a combination thereof.

The polymers of the second polymer component may be selected from any biocompatible polymers capable of forming or imparting certain characteristics to the hydrogel composites and compositions described herein. The polymers of the second polymer component, for example, may be selected from at least one biopolymer, polyamine, or a combination thereof.

In one embodiment, the second polymer component includes a second polymer that is a biopolymer having one or more amines, such as primary amines, secondary amines, or a combination thereof. Non-limiting examples of biopolymers include chitosan, collagen, gelatin, other structural biomolecules, or a combination thereof. In a particular embodiment, the second polymer comprises a polyamine. The polyamine may be synthetic. Non-limiting examples of polyamines include amine-terminated, multi-arm poly(ethylene oxide) and polyethyleneimine. In another embodiment, the second polymer component includes a second polymer that comprises both (i) a biopolymer having one or more amines, and (ii) a polyamine. Therefore, as used herein, the phrase “second polymer” refers to the one or more polymers of the second polymer component that include one or more functional groups, e.g., amines, that are capable of reacting with the one or more functional groups of the first polymer component, such as aldehydes.

In some embodiments, the second polymer is a commercially available amine-terminated polymer, such as Type I collagen, Type II collagen, Type III collagen, gelatin that is acid- or base-catalyzed (i.e., Type A or Type B), or 10 kD dextran (Pharmacosmos A/S, Denmark).

### Dendrimer Component

In embodiments, the second solution comprises a dendrimer component. The dendrimer component of the second solution may include [1] the first dendrimer to which a drug is conjugated, [2] a second dendrimer as provided herein, or [3] the first dendrimer to which a drug is conjugated and a second dendrimer as provided herein. The second dendrimer may be substituted with one or more functional groups, such as amines, that are capable of reacting with the one or more functional groups of the first polymer of the first polymer component.

In some embodiments, the second dendrimer has amines on at least a portion of its surface groups, which are commonly referred to as “terminal groups” or “end groups.” The second dendrimer may have amines on from 25% to 100% of its surface groups. In some embodiments, the second dendrimer has amines on 100% of its surface groups. In one embodiment, the second dendrimer has amines on less than 75% of its surface groups. As used herein, the term “second dendrimer” refers to any compound with a polyvalent core covalently bonded to two or more dendritic branches. In some embodiments, the polyvalent core is covalently bonded to three or more dendritic branches. In one embodiment, the amines are primary amines. In another embodiment, the amines are secondary amines. In yet another embodiment, one or more surface groups have at least one primary and at least one secondary amine.

In one embodiment, the second dendrimer extends through at least 2 generations. In another embodiment, the second dendrimer extends through at least 3 generations. In yet another embodiment, the second dendrimer extends through at least 4 generations. In still another embodiment, the second dendrimer extends through at least 5 generations. In a further embodiment, the second dendrimer extends through at least 6 generations. In still a further embodiment, the second dendrimer extends through at least 7 generations.

In one embodiment, the second dendrimer has a molecular weight of about 1,000 to about 1,000,000 Daltons. In a further embodiment, the second dendrimer has a molecular weight of about 3,000 to about 120,000 Daltons. In another embodiment, the second dendrimer has a molecular weight of about 10,000 to about 100,000 Daltons. In yet another embodiment, the second dendrimer has a molecular weight of about 20,000 to about 40,000 Daltons. Unless specified otherwise, the “molecular weight” of the second dendrimer refers to the number average molecular weight.

Generally, the second dendrimer may be made using any known methods. In one embodiment, the second dendrimer is made by oxidizing a starting second dendrimer having surface groups comprising at least one hydroxyl group so that at least a portion of the surface groups comprise at least one amine. In another embodiment, the second dendrimer is made by oxidizing a starting generation 5 (G5) second dendrimer having surface groups comprising at least one hydroxyl group so that at least a portion of the surface groups comprise at least one amine. In yet another embodiment, the second dendrimer is made by oxidizing a starting G5 second dendrimer having surface groups comprising at least one hydroxyl group so that about 25% to 100% of the surface groups comprise at least one amine. In a particular embodiment, the second dendrimer is a G5 second dendrimer having primary amines on about 25% to 100% of the second dendrimer's surface groups. In a certain embodiment, the second dendrimer is a G5 second dendrimer having primary amines on about 25% of the second dendrimer's surface groups.

In one embodiment, the second dendrimer is a poly(amidoamine)-derived (PAMAM) second dendrimer. In another embodiment, the second dendrimer is a G5 PAMAM-derived second dendrimer. In yet another embodiment, the second dendrimer is a G5 PAMAM-derived second dendrimer having primary amines on about 25% to 100% of the second dendrimer's surface groups. In a further embodiment, the second dendrimer is a G5 PAMAM-derived second dendrimer having primary amines on about 25% of the second dendrimer's surface groups.

In one embodiment, the second dendrimer is a poly(propyleneimine)-derived second dendrimer.

In some instances, at least one of the first solution, the first polymer component, the second solution, the second polymer component, and the second dendrimer further includes one or more additives. Generally, the amount of additive may vary depending on the application, tissue type, concentration of the second dendrimer in the second solution, the type of second dendrimer, concentration of the second polymer component in the second solution, the type of second polymer component, the type of first polymer component, and/or the concentration of the first polymer component in the first solution. Example of suitable additives, include but are not limited to, pH modifiers, thickeners, antimicrobial agents, colorants, surfactants, and radio-opaque compounds. Specific examples of these types of additives are described herein. In one embodiment, at least one of the first solution, the first polymer component, the second solution, the second polymer component, and the second dendrimer comprises a foaming additive.

In particular embodiments, at least one of the first solution, the first polymer component, the second solution, the second polymer component, and the second dendrimer includes one or more drugs. In such embodiments, the hydrogel composites or compositions may serve as a matrix material for controlled delivery of the one or more drugs. The one or more drugs may be essentially any pharmaceutical agent suitable for local, regional, or systemic administration from a quantity of the hydrogel composite or composition that has been applied to one or more tissue sites in a patient. In one embodiment, the one or more drugs comprises a thrombogenic agent. Non-limiting examples of thrombogenic agents include thrombin, fibrinogen, homocysteine, estramustine, and combinations thereof. In another embodiment, the one or more drugs comprises an anti-inflammatory agent. Non-limiting examples of anti-inflammatory agents include indomethacin, salicyclic acid acetate, ibuprophen, sulindac, piroxicam, naproxen, and combinations thereof. In still another embodiment, the one or more drugs comprises an anti-neoplastic agent. In still other embodiments, the one or more drugs is one for gene therapy. For example, the one or more drugs may comprise siRNA molecules to combat cancer. In a particular embodiment, the one or more drugs comprises human bone morphogenetic protein 2. Other drugs are envisioned.

In other particular embodiments, at least one of the first solution, the first polymer component, the second solution, the second polymer component, and the second dendrimer includes one or more cells. For example, in any of these embodiments, the hydrogel composites or compositions may serve as a matrix material for delivering cells to a tissue site at which the hydrogel composites or compositions have been applied. In embodiments, the cells may comprise endothelial cells (EC), endothelial progenitor cells (EPC), hematopoietic stem cells, or other stem cells. In one embodiment, the cells are capable of releasing factors to treat cardiovascular disease and/or to reduce restenosis. Other types of cells are envisioned.

### Formation of Hydrogel Composites and Compositions

Generally, the hydrogel composites and compositions described herein may be formed by combining the first solution and the second solution in any manner. In some embodiments, the first solution, and the second solution are combined before contacting a biological tissue. In other embodiments, the first solution, and the second solution are combined, in any order, on or in a biological tissue. In further embodiments, the first solution is applied to a first biological tissue, the second solution is applied to a second biological tissue, and the first and second biological tissues are contacted. In still a further embodiment, the first solution is applied to a first region of a biological tissue, the second solution is applied to a second region of a biological tissue, and the first and second regions are contacted.

Generally, the hydrogel composites and compositions may be applied to a biological tissue as a drug delivery composition. The hydrogel composites and compositions also may be configured as a tissue adhesive or sealant.

The hydrogel composites and compositions may be applied to the biological tissue using any suitable tool and methods. Non-limiting examples include the use of syringes or spatulas. Double barrel syringes with rigid or flexible discharge tips, and optional extension tubes, known in the art are envisioned.

As used herein, the hydrogel composites and compositions are a “treatment” when they improve the response of at least one biological tissue to which they are applied. In some embodiments, the improved response is slowing or reversing tumor growth, inducing cytotoxicity in cancer cells, lessening overall inflammation, improving the specific response at the wound site/interface of the tissue and hydrogel composites or compositions, enhancing healing, repairing torn or broken tissue, or a combination thereof. As used herein, the phrase “lessening overall inflammation” refers to an improvement of histology scores that reflect the severity of inflammation. As used herein, the phrase “improving the specific response at the wound site/interface of the tissue and hydrogel composite or compositions” refers to an improvement of histology scores that reflect the severity of serosal neutrophils. As used herein, the phrase “enhancing healing” refers to an improvement of histology scores that reflect the severity of serosal fibrosis.

In embodiments, the hydrogel composites and compositions may be used in challenging or awkward implantation environments, including under flowing liquids and/or in inverted geometries.

Before or after contacting one or more biological tissues, the hydrogel composites and compositions may be allowed adequate time to cure or gel. When the hydrogel composites and compositions “cure” or “gel,” as those terms are used herein, it means that the one or more functional groups of the first polymer have undergone one or more reactions with the dendrimer and/or second polymer, and one or more biological tissues. Not wishing to be bound by any particular theory, it is believed that the hydrogel composites and compositions described herein are effective because the first polymer component reacts with both (i) the dendrimer and/or second polymer component, and (ii) the surface of the biological tissues. In certain embodiments, the first polymer component's aldehyde functional groups react with the amines on (i) the dendrimer and/or second polymer component, and (ii) the biological tissues to form imine bonds. In these embodiments, it is believed that the amines on the dendrimer and/or second polymer component react with a high percentage of the aldehydes of the first polymer component, thereby reducing toxicity and increasing biocompatibility of the hydrogel composites and compositions. Typically, the time needed to cure or gel the hydrogel composites and compositions will vary based on a number of factors, including, but not limited to, the characteristics of the first polymer component, second polymer component and/or dendrimer, the concentrations of the first solution and second solution, the pH of the first and second solution, and the characteristics of the one or more biological tissues. In embodiments, the hydrogel composites and compositions will cure sufficiently to provide desired bonding or sealing shortly after the components are combined. The gelation or cure time should provide that a mixture of the components can be delivered in fluid form to a target area before becoming too viscous or solidified and then once applied to the target area sets up rapidly thereafter. In one embodiment, the gelation or cure time is less than 120 seconds. In another embodiment, the gelation or cure time is between 3 and 60 seconds. In a particular embodiment, the gelation or cure time is between 5 and 30 seconds.

### Tissue Specific Formulations

Generally, the hydrogel composites and compositions may be adjusted in any manner to compensate for differences between tissues. In one embodiment, the amount of first polymer component is increased or decreased while the amount of dendrimer and/or second polymer component is unchanged. In another embodiment, the amount of dendrimer and/or second polymer component is increased or decreased while the amount of first polymer component is unchanged. In another embodiment, the concentration of the first polymer component in the first solution is increased or decreased while the second solution is unchanged. In yet another embodiment, the concentration of the dendrimer and/or second polymer component in the second solution is increased or decreased while the first solution is unchanged. In a further embodiment, the concentrations of the both the first polymer component in the first solution and the dendrimer and/or second polymer component in the second solution are changed.

When the amine density on the surface of a particular biological tissue is unknown due to disease, injury, or otherwise, an excess of the first solution may, in some embodiments, be added when the hydrogel composites and compositions are first applied, then the amount of first solution may be reduced, e.g., incrementally or drastically, until the desired effect is achieved. The “desired effect,” in this embodiment, may be an appropriate or adequate curing time, adhesion, sealing, treatment, drug delivery, or a combination thereof. Not wishing to be bound by any particular theory, it is believed that an excess of the first solution may be required, in some instances, to obtain the desired effect when the amine density on a biological tissue is low. Therefore, adding an excess will help the user, in this embodiment, achieve adequate sealing or adhesion or treatment in less time.

In other embodiments, however, a lower amount of the first solution may be added when the hydrogel composites and compositions are first applied, then the amount of first solution may be increased, e.g., incrementally or drastically, until the desired effect is achieved, which may be adequate curing time, adhesion, sealing, treatment, or a combination thereof.

In embodiments, the hydrogel composites and compositions can be optimized in view of a target biological tissue, by adjusting one or more of the following: rheology, mechanics, chemistry/adhesion, degradation rate, drug delivery, and bioactivity. These can be adjusted, in embodiments, by altering the type and/or concentration of the first dendrimer to which a drug and binding peptide are conjugated, the type and/or concentration of the first polymer component, and type and/or concentration of the dendrimer, the type and/or concentration of the second polymer component, or a combination thereof.

### Hydrogel Composite and Composition Kits

In another aspect, a kit is provided that comprises a first part that includes the first solution, and a second part that includes the second solution. The kit may further include an applicator or other device means, such as a multi-compartment syringe, for storing, combining, and delivering the two solutions and/or the resulting hydrogel composites and compositions to a tissue site.

In one embodiment, the kit comprises separate reservoirs for the first solution and the second solution. In certain embodiments, the kit comprises reservoirs for first solutions of different concentrations. In other embodiments, the kit comprises reservoirs for second solutions of different concentrations.

In one embodiment, the kit comprises instructions for selecting an appropriate concentration or amount of at least one of the first solution and/or second solution to compensate or account for at least one characteristic of one or more biological tissues. In one embodiment, the hydrogel composites and compositions are selected based on one or more predetermined tissue characteristics. For example, previous tests, may be performed to determine the number of density of bonding groups on a biological tissue in both healthy and diseased states. Alternatively, a rapid tissue test may be performed to assess the number or density of bonding groups. Quantification of tissue bonding groups can be performed by contacting a tissue with one or more materials that (1) have at least one functional group that specifically interacts with the bonding groups, and (2) can be assessed by way of fluorescence or detachment force required to separate the bonding groups and the material. In another embodiment, when the density of bonding groups on a biological tissue is unknown, an excess of the first polymer having one or more aldehydes, may be initially added as described herein to gauge the density of bonding groups on the surface of the biological tissue.

In certain embodiments, the kit comprises at least one syringe. In one embodiment, the syringe comprises separate reservoirs for the first solution and second solution. The syringe may also comprise a mixing tip that combines the two solutions as the plunger is depressed. The mixing tip may be release-ably securable to the syringe (to enable exchange of mixing tips), and the mixing tip may comprise a static mixer. In some embodiments, the reservoirs in the syringe may have different sizes or accommodate different volumes of solution. In other embodiments, the reservoirs in the syringe may be the same size or accommodate the same volumes of the solution.

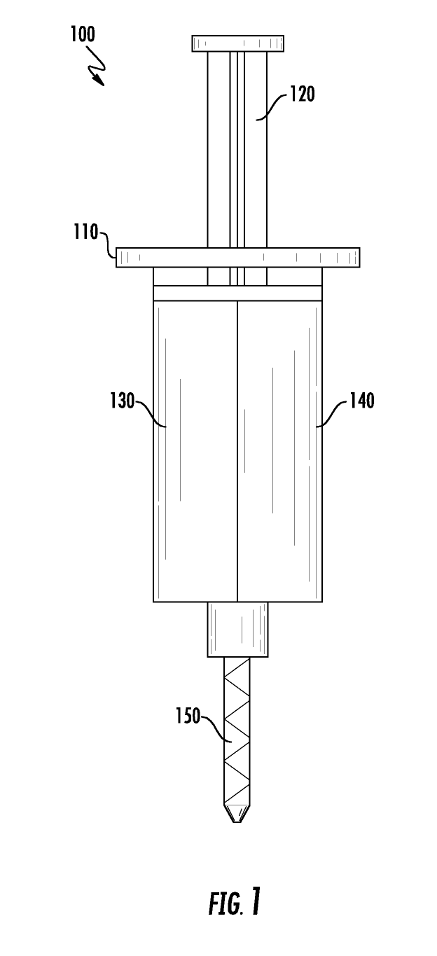

FIG. 1 depicts one embodiment of a syringe 100. The syringe 100 includes a body 110 with two reservoirs (130, 140). A first solution is disposed in the first reservoir 130, and a second solution is disposed in the second reservoir 140. The two reservoirs (130, 140) are emptied by depressing the plunger 120, which pushes the contents of the two reservoirs (130, 140) into the mixing tip 150 and out of the syringe 100.

In a further embodiment, one or more of the reservoirs of the syringe may be removable. In this embodiment, the removable reservoir may be replaced with a reservoir containing a first solution or second solution of a desired concentration.

In a preferred embodiment, the kit is sterile. For example, the components of the kit may be packaged together, for example in a tray, pouch, and/or box. The packaged kit may be sterilized using known techniques at suitable wavelengths (where applicable), such as electron beam irradiation, gamma irradiation, ethylene oxide sterilization, or other suitable techniques.

In the descriptions provided herein, the terms “includes,” “is,” “containing,” “having,” and “comprises” are used in an open-ended fashion, and thus should be interpreted to mean “including, but not limited to.” When methods and composite materials are claimed or described in terms of “comprising” various components or steps, the composite materials and methods can also “consist essentially of” or “consist of” the various components or steps, unless stated otherwise.

The terms “a,” “an,” and “the” are intended to include plural alternatives, e.g., at least one. For instance, the disclosure of “a drug,” “a binding peptide,” “a first dendrimer”, and the like, is meant to encompass one, or mixtures or combinations of more than one, drug, binding peptide, first dendrimer, and the like, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate. As a representative example, Applicant discloses, in one embodiment, an average of about 6 to about 10 molecules of the drug are conjugated to each molecule of the first dendrimer. This range should be interpreted as encompassing an average number of molecules in a range of about 6 to about 10, and further encompasses “about” each of 7, 8, and 9, including any ranges and sub-ranges between any of these values.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

### Example 1—Dendrimer-Peptide Conjugates

Two conjugates were made by conjugating fluorescently labeled PAMAM dendrimer generation 5 to either EGF or EGFmp through a PEGylated amine-to-sulfhydryl crosslinker, specifically SM(PEG)₂. The EGFmp was modified with four glycine residues terminated with a cysteine to allow for sulfhydryl-maleimide conjugation.

PAMAM dendrimer (Dendritech) was fluorescently tagged with either AlexaFluor® 594 carboxylic acid, succinimidyl ester (Life Technologies) or 6-(fluorescein-5-(and-6-)-carboxyamido)hexanoic acid, succinimidyl ester (Life Technologies) in DMF at a concentration of 10 mg/mL in 50 mM bicarbonate buffer (pH 8.5). Fluorescent dendrimers were dialyzed against double-distilled water (ddH₂O) overnight in 10 kDa MWCO dialysis cassettes (Thermo Scientific) and lyophilized. Tagged dendrimers were dissolved in ddH₂O water to a final concentration of 500 μM and characterized by fluorescence spectroscopy.

The PEGylated crosslinker density on the surface of the dendrimer was determined by Ellman's assay by reacting an excess cysteine to the terminal maleimide groups and measuring the unreacted excess cysteine.

Dendrimer EGF conjugation: 1 mg of human recombinant EGF (Peprotech) was dissolved in 0.1M phosphate buffer with 0.15M NaCl at a concentration of 5 mg/ml and 22.2 ul of TCEP 20 mM were added to reduce the disulfide bonds. The reaction crude was stirred for two hours at room temperature. Then, the reaction was dialyzed twice in a 3 kDa MWCO centrifugal filter (Centricon, Millipore) at 4000 RCFs for 15 minutes at room temperature. In parallel, 1 mg of 500 μM fluorescently tagged PAMAM dendrimer was dissolved in 100 μl of 0.1M phosphate buffer and 831 μl of a 20 mM SM(PEG)₂ solution was added dropwise. The mix was allowed to react for 30 minutes and dialyzed twice in a 10 kDa centrifugal filter (Centricon, Millipore) at 4000 RCFs for 15 minutes at room temperature. Next, the reduced EGF solution was mixed with the dendrimer conjugated with the linker and stirred at room temperature for 4 hours. The reaction product was dialyzed for two hours and overnight against PBS. The purified dendrimer-EGF conjugate's concentration was determined by fluorescence spectroscopy and the structure characterized by UV-VIS spectroscopy at 275 nm (EGF/dendrimer ratio).

Dendrimer-EGFmp conjugation: 1 mg of 500 μM fluorescently tagged PAMAM dendrimer was dissolved in 100 μl of 0.1M phosphate buffer and 831 μl of a 20 mM SM(PEG)₂ solution was added dropwise. The mix was allowed to react for 30 minutes and dialyzed twice in a 10 kDa centrifugal filter (Centricon, Millipore) at 4000 RCFs for 15 minutes at room temperature. Next, 610 μl of peptide solution (4 mg/ml in 0.1M phosphate buffer; Sequence: NH₂—CGGGGAEYLR-COOH, Han, C. Y. et al. Int J Nanomedicine 8, 1541-1549 (2013)) were added dropwise to the dendrimer conjugated with the linker and stirred at room temperature for 4 hours. The reaction product was dialyzed for two hours and overnight against PBS. The purified dendrimer-EGFpep conjugate's concentration was determined by fluorescence spectroscopy and the structure determined by Ellman's assay and UV-VIS spectroscopy at 275 nm (peptide/dendrimer ratio).

Successful peptide and EGF conjugation was corroborated by UV-VIS spectroscopy, which showed an increase in absorbance at 280 nm, which is believed to originate from the aromatic amino acids present both in the peptide and EGF. The ligand to dendrimer ratios were calculated as the quotient EGF or peptide concentrations (measured from their absorbance contributions at 280 nm) and dendrimer concentration (determined from the absorbance of the fluorescent tag at 594 nm), as well as by Ellman's assay of the unreacted EGF or peptide. Specifically, NanoDrop 2000c (Thermo Scientific, Tewksbury, Mass.) was used to characterize the dendrimer conjugates. Absorbance at 275 nm was used to determine the ratio of EGF or peptide to dendrimer.

The substantially lower molecular weight and volume of EGFmp (1.1 kDa, linear sequence) with respect to EGF (6.2 kDa, tertiary bulky structure), allowed for a higher peptide conjugation efficiency per dendrimer (˜40 peptides) compared to that of EGF (˜5 proteins).

The foregoing is believed to demonstrate that the dendrimer-peptide conjugates of this example were stable, and permitted a relatively high peptide density on their surfaces.

Dynamic Light Scattering (DLS) was employed to report on the size of the conjugates. Both dendrimer-EGF and dendrimer-EGFmp showed an increase in hydrodynamic diameter with respect to the naked dendrimer (mean diameters of 6.18±1.33, 9.46±0.98, and 7.31±1.16 for naked dendrimer, dendrimer-EGF, and dendrimer EGFmp, respectively). The samples were diluted to a concentration around 10 μM in PBS and the dendrimer complexes' sizes were determined by a laser DLS method using a DynaPro Plate Reader instrument (Wyatt Technology Corp., USA).

Even though both conjugates had roughly the same size, the dendrimer-EGF conjugates appeared to form aggregates of 9.86±1.69 μm in diameter. Moreover, DLS studies of dendrimer-EGF conjugates a week post-reaction showed an increase in size distribution of the main peak at 9.46 nm, as corroborated by the increase in standard deviation, as well as the appearance of other peaks of varying sizes—63.16±13.17 nn, 612.33±94.44, and 4.59±0.64 μm—which are believed to indicate conjugate degradation.

After one week, the naked dendrimers also formed aggregates that could be dissolved by sonication, and, as a result, are believed to be formed by electrostatic interactions. High-resolution cryo-TEM showed discrete naked dendrimer and dendrimer-EGFmp particles of 4.2 nm and 5.6 nm in diameter, respectively. The dendrimer-peptide was demonstrated to be more stable in solution after a week than dendrimer-EGF, which showed an increase in size distribution. Images of dendrimer-EGF conjugates showed two distinct populations; aggregates and discrete particles of 8.7 nm in diameter. This is believed to [1] correlate with the observations gathered from DLS experiments regarding the formation of aggregates, and [2] corroborate the lower stability in solution of dendrimer-EGF conjugates.

### Example 2—Pathway Activation

The dendritic pro-drug of Example 1 was studied to determine whether it is capable of triggering the EGFR signaling pathway through its interaction with the EGF receptor. EGFR signaling pathway is believed to induce growth, differentiation, migration, adhesion, and cell survival through various interacting signaling pathways. It is believed to be critical for the design of chemotherapeutic pro-drugs utilizing this receptor to ensure this pathway was not triggered, leading to further cancer cell proliferation and survival, which could counteract the effects of the chemotherapeutic drug.

Western Blot studies showed that full EGF, both on its own and conjugated to the dendrimer, triggered EGFR pathway, as shown by the presence of a band corresponding to the phosphorylated form or EGFR. On the contrary, the synthetic peptide did not elicit EGFR phosphorylation when cells were dosed with the free peptide or the dendritic conjugate. Moreover, the number of peptide copies (20 or 60 copies) conjugated to the dendrimer did not affect EGFR phosphorylation.

These results are believed to corroborate one of the advantages of using synthetic peptides over full proteins, namely, the ability to be synthesized to interact avidly with receptors without eliciting the response of the actual biological ligand.

### Example 3—Affinity of EGF and EGFmp for Receptor

The relative affinity of EGF and EGFmp for the receptor was studied to determine whether the peptide was an antagonist that could potentially block the receptor from EGF-mediated activation. EGFR-overexpressing cancer cells were incubated with a mix of EGF and peptide and EGFR activation was also assessed by Western Blot.

The results showed that the EGFmp did not behave as an EGF antagonist, as evidenced by the phosphorylated EGFR bands observed, regardless of the relative concentrations of growth factor and peptide.

Specifically, images of Western Blot gels showed bands corresponding to phosphorylated EGFR when cells were incubated with (a) free EGF, but none were observed with (b) free peptide. The corresponding conjugates followed the same trend with dendrimer-EGF but not dendrimer EGF-mp activating the pathway. The EGF mimicking peptide did not block EGF-mediated EGFR pathway activation. Actin was used as an internal control in all gels.

### Example 4—Cellular Uptake of Dendrimer-Peptide Conjugates

The effects of the peptide type (i.e., full growth factor EGF versus EGFR-binding synthetic peptide) on cellular uptake via receptor-mediated endocytosis were determined.

The dendrimers conjugated with multiple copies of a ligand for EGFR enhanced RME in cancer cells, and it is believed that this result was due to the high density of receptors that prompted numerous stimulation points. The dendrimers also did not elicit RME in healthy cells, owing to their basal levels of EGFR.

To validate the selectivity of the conjugates towards EGFR overexpressing cancer cells, the uptake of the conjugates and therapeutic effect in a breast cancer cell line overexpressing the EGF receptor (MDA-MB-468) and in a healthy mammary epithelial cell line (HMEpC) were determined. The differential receptor expression between these two cell lines by immunostaining and Western Blot against EGFR were corroborated. EGFR expression was quantified by reverse transcription polymerase chain reaction (RT-qPCR) and it was found that MDA-MB-468 cells expressed more EGFR (2-fold) than HMEpC.

MDA-MB-468 (ATCC) human breast cancer cells were cultured and maintained in Advanced DMEM medium supplemented with 2% FBS and 1% penicillin/streptomycin/glutamine. Prior to experimentation, cells were grown to 80% confluence on 100-mm plates in an environment at 37° C. with 5% CO₂. HMEpC (Lonza) cells were cultured and maintained in MEGM medium supplemented with 100 ng/ml Cholera Toxin. Prior to experimentation, cells were grown to 80% confluence on 100-mm plates in an environment at 37° C. with 5% CO₂.

EGFR-overexpressing MDA-MB-468 cells were incubated with fluorescently labeled naked dendrimer, dendrimer-EGF, or dendrimer-EGFmp at 37° C. or 4° C. for 5 hours to assess intracellular uptake. Uptake at 4° C. was conducted to examine whether uptake occurred via energy-independent processes, as energy-dependent processes such as RME are inactive at 4° C. The conjugates' uptake was differentially reduced as a function of treatment.

For the uptake experiment at 37° C., MDA-MB-468 and HMEpC cells were seeded in their respective growing media at a density of 30·10³ cells/well in a 96 well plate and incubated 24 h at 37° C. and 5% CO₂. Incubation medium was removed and replaced by complete L15 medium containing either naked dendrimer or dendrimer complexes at a concentration of 10 μM. Complete L15 medium was used as a negative control. After 5 h of incubation with the dendrimer complexes, cells were washed with PBS, fixed with PFA 4% and nuclei were stained with DAPI. Samples were analysed by fluorescence microscopy (Nikon Eclipse Ti-E) and images were processed using ImageJ software. For the uptake experiment at 4° C., cells were cultured and treated as described above after 1 hour pre-incubation at 4° C. All dendrimer solutions were also cooled down prior to performing the experiment.

Despite shutting down energy-dependent uptake mechanisms, naked dendrimers' internalization at 4° C. was still evident, which is believed to indicate both energy-dependent and independent internalization mechanisms.

Nanoscale hole formation is thought to take place through interactions between the positive charges on the surface of the dendrimer and the cell membrane. Indeed, high uptake of naked dendrimer was observed in HMEpC, likely due to nanoscale hole formation, which is believed to be minimized in the case of the modified dendrimer conjugates.

Taken together, these data are believed to suggest that both dendrimer-EGF and dendrimer-EGFmp were being uptaken into cells through energy-dependent mechanisms selected to the cancer cells under study.

Flow cytometry data is believed to corroborate the energy-independent mechanism of dendrimer conjugates. While naked dendrimer uptake was extensive in EGFR overexpressing cells at 37° C. (mean fluorescence/cell 1.12×10⁵±9.1×10⁴), dendrimer conjugates showed a more moderate uptake (mean fluorescence/cell dendrimer-EGFmp 5.1×10³±5.4×10³ and dendrimer-EGF 2×10³±2.9×10³).

Uptake at 4° C. was reduced in all cases; however, the decrease was more significant in the case of the dendritic conjugates, with levels of uptake comparable to those of the untreated controls (mean fluorescence/cell dendrimer-EGFmp 40±40; dendrimer-EGF 1.4×10²±4.5×10² and untreated control 43±43). Naked dendrimer uptake was significantly reduced at 4° C. compared to that at 37° C., but still maintained high levels compared to its conjugated counterparts (1.7×10⁴±1.8×10⁴ at 4° C. compared to 1.1×10⁵±9.1×10⁴ at 37° C.), which is believed to corroborate that the dendrimers were internalized via a combination of mechanisms, both energy-dependent and independent.

Dendrimer conjugates showed a decrease in uptake in HMEpC, with more pronounced reduction in the case of dendrimer-EGFmp (dendrimer-EGFmp 5.1×10²±4.9×10² and dendrimer-EGF 9.4×10²±8.2×10²). This is believed to suggest that dendrimer-EGFmp was being internalized solely through energy-dependent mechanisms more specific to MDA-MB-468 breast cancer cells than healthy HMEpC.

Also corroborated was the observation that the energy-dependent uptake by the dendritic conjugates was EGFR-mediated. For this purpose, EGFR was blocked with a neutralizing antibody prior to MDA-MB-468 incubation with different dendrimers. Naked dendrimer and dendrimer-EGF uptake were not altered by receptor neutralization, while dendrimer EGFmp internalization was completely abrogated. This is believed to indicate exclusive EGFR-mediated uptake. The indiscriminate uptake of naked dendrimer was observed to be independent of cell type and treatment. The data observed by epi-fluorescence microscopy correlated with the flow cytometry results.

Neutralizing antibody: MDA-MB-468 were seeded at a density of 30·10³ cells/well in a 96 well plate and incubated 24 h at 37° C. and 5% CO₂. Incubation medium was removed and replaced by complete L15 culture medium containing anti-EGFR neutralizing antibody clone LA1 (Millipore) at a concentration of 30 μg/ml. Cells were incubated at 37° C. for 1 hour, after which either naked dendrimer or dendrimer complexes were added to a final concentration of 10 μM. After 5 h of incubation with the dendrimer complexes, cells were washed with PBS and fixed with PFA 4%. Nucleuses were stained after fixation with DAPI. Samples were analysed by fluorescence microscopy (Nikon Eclipse Ti-E) and images were processed using ImageJ software.

Also investigated was whether the dendrimer-EGF non-specific uptake following EGFR neutralization was due to alternative EGF uptake mechanisms or a result of the chemical modification during the conjugation process. To examine the specificity of EGF uptake through EGFR, MDA-MB-468 cells were incubated with free, labeled EGF. These cells internalized EGF in the absence of neutralizing antibody.

However, a total abrogation of EGF uptake was observed when the receptor was inactivated, which is believed to indicate its specificity for the EGF receptor. It is believed that EGF modifications during the conjugation reaction could alter EGF tertiary structure, potentially activating other internalization mechanisms. TCEP-treated EGF was indeed internalized after blocking EGFR, but to a lesser extent than without receptor neutralization.

Also determined was whether incomplete EGF coverage of the PEGylated dendrimer may lead to non-specific uptake through other internalization mechanisms. It was observed that the intermediate PEGylated dendrimer and the dendrimer-EGF had comparable levels of cellular internalization, as determined by flow cytometry.

Prior to performing the RT-qPCR, cells were seeded at a density of 25.10⁴ cells/well in a 24 well-plate and incubated at 37° C. with 5% CO₂ for 72 h. RNA was isolated using RNeasy Mini kit (QIAGEN). RNA concentration was determined by spectrophotometry using NanoDrop 2000c (Thermo Scientific, Tewksbury, Mass.). For each sample, 0.1 μg of total RNA was used for cDNA synthesis, and the reverse transcription reaction was performed with a High Capacity cDNA Reverse Transcription kit (Applied Biosystems) according to the manufacturer's instructions. Real-time polymerase chain reaction (qPCR) was performed using the TaqMan Universal Master Mix II (Applied Biosystems) and the TaqMan probes (EGFR assay ID: Hs_, GAPDH assay ID:) in a LightCycler® 480 Real-Time PCR System (Roche). The relative expression mRNA level of EGFR was computed and housekeeping gene glyceraldehyde 3-phosphate dehydrogenase (GAPDH) was used as the endogenous control to normalize for variations in the quality of RNA and the amount of input cDNA, as described previously. qPCR steps included a preincubation step for 5 min at 95° C., followed by 40 cycles of three steps: 10 s at 95° C., 20 s at 60° C., and 30 s at 72° C. The threshold cycle (Ct) values were generated automatically by the LightCycler 480 software, version 1.5, and the Ct comparative method for mRNA level quantification was calculated.

Immunostaining of in vitro samples: MDA-MB-468 and HMEpC cells were seeded at a density of 30·10³ cells/well in a 96 well-plate and incubated 24 h at 37° C. and 0.8% CO₂. Immunostaining against EGFR of both cell lines was performed following standard protocols. EGF receptor (D38B1) XP(R) Rabbit mAb (Cell Signalling) was used as primary antibody and Goat Anti-Rabbit IgG conjugated to Alexa Fluor 488 (Thermo Fisher Scientific) was used as secondary antibody. Cell nuclei were stained with DAPI. Samples were analysed by fluorescence microscopy (Zeiss Axiovert 200M) and images were processed using ImageJ software.

### Example 5—Cytocidal Effect and Intracellular Fate

Also investigated was the cancer-specific cytocidal effect associated with a dendritic pro-drug, both in healthy and cancer cells.

The dendritic pro-drug molecule was based on the dendrimer conjugate of Example 1, and, therefore, was a PAMAM-dendrimer generation 5 core decorated with synthetic EGFR-binding peptides that could elicit RME selectively in EGFR over-expressing cancer cells. The dendrimer to which the binding peptide was conjugated was a PAMAM dendrimer, generation 5, with amines on 100% of its surface groups, and a molecule weight of 28,826 Da.

30 mg of doxorubicin (Cayman Chemicals) were dissolved in ddH₂O to yield a 10 mg/mL solution, the pH was adjusted to 8-9 with 0.25 M NaOH and the solution cooled down in an ice-water bath. Aconitic anhydride (600 μL, 100 mg/mL in dioxane; Sigma-Aldrich) was added dropwise while simultaneously adjusting the pH to 8-9 with NaOH 0.25 M. The mixture was allowed to react for 20 minutes in the ice-water bath, then diluted with 3 mL of ice-cold ddH₂O and precipitated with ice-cold 1 M hydrochloric acid until pH 2-3 for 30 minutes. The product was centrifuged for 5 minutes at 2500 RPMs at 4° C. and the supernatant discarded. The precipitate was then redissolved in 5 mL of ddH2O and the minimum amount of NaOH 0.25 M, cooled down and re-precipitated in 1 M hydrochloric acid as described above. The final pure product was dissolved in DMF:DMSO (3:1 ratio) for further use. The product was characterized by UV-Vis spectroscopy at 490 nm.

19 mg of maleimide-PEG2-succinimidyl ester (SM(PEG)₂) were dissolved to 1.00 mL with phosphate buffer 0.1 M. This solution was added dropwise over 10 mg PAMAM dendrimer generation 5 (100% amines) diluted to 1.00 mL with phosphate buffer 0.1 M, and allowed to react for 30 min. To the reaction crude, 61 μl of 10 mg/ml cysteine in phosphate buffer 0.1 M with EDTA 0.1 M were added and allowed to react for 1 hour. In parallel, dox-aconityl was activated with EDC and NHS for 30 min, and then added dropwise to the dendrimer-cysteine (Dend-Cys-NH2) solution with a ratio of 14 ptmole Dox-COOH/1 μmole Dend-Cys-NH₂ and the reaction was carried out for 1 hour. The reaction crude was purified using PD-10 desalting columns (GE Healthcare PD-10 prepacked desalting columns), previously equilibrated with 50 mM phosphate buffer. 2 ml of cysteine-peptide (4 mg/mL in 0.1 M phosphate buffer with 0.1 M EDTA) were added to the purified product and allowed to react 90 min. The final product was dialysed overnight with a 10 kDa MWCO membrane (Thermo Scientific) against PBS ×1.

UV-VIS studies of the conjugates showed successful conjugation of an average of 8 doxorubicin molecules per dendrimer molecule.

Also, at equal concentrations of doxorubicin measured by UV-VIS, fluorescence of the drug conjugated to the dendrimer decreased 9-fold compared to that of the free drug. The observed fluorescence quenching is believed to be most likely caused by the local basic pH imparted by the dendrimer, as doxorubicin fluorescence had a strong dependence with pH. A 12-fold decrease in doxorubicin's fluorescence between 5.52 and 10.40 has been reported, which is believed to further corroborate the observed findings in the dendritic pro-drug (Karukstis, K. et al. Biophys. Chem. 1998; 73(3): 249-63).

Dendrimer-conjugated doxorubicin's fluorescence quenching was used to study pH-triggered release. In vitro release data at pH 7.4 and 5.5 did not show any changes in fluorescence intensity over the course of 48 hours compared to free doxorubicin-aconityl, which is believed to suggest that the pH-sensitive aconityl linker was not cleaved at acidic pH, as previously reported. It was then investigated whether doxorubicin could be released in a cellular environment, in which there are more elements at play than just pH (e.g., proteases). MDA-MB-468 were incubated with dendritic pro-drug and the fluorescence intensity was followed over time.

Fluorescence microscopy images showed a buildup of dendritic pro-drug on the surface of the cells after a 2-hour incubation, and accumulation in the nucleus at 6 and 24 hours of incubation, with no concomitant increase in fluorescence. These results are believed to suggest that doxorubicin was not cleaved in the intracellular environment, and that the intact dendritic pro-drug was capable of penetrating the nuclear membrane and accumulating in the nucleus.

Also investigated was the endosomal escape mechanism of the dendritic pro-drug by tracking early and late endosomes and lysosomes over time. Dendritic pro-drug was also tracked owing to its inherent autofluorescence. Early and late endosome and lysosome tracking of dendritic pro-drug in MDA-MB-468 cells was studied. Endosomes were labeled prior to pro-drug incubation with CellLight Early or Late Endosomes-GFP, BacMam 2.0 and lysosomes were stained with LysoTracker Green DND-26 after cells fixation.

At early time points (4 and 8 hours), the presence of early endosomes was evidenced by the green fluorescence observed. At 8 hours incubation, the fluorescence intensity corresponding to late endosomes increased mildly with respect to that of 4 hours, which is believed to indicate that some of the dendritic pro-drug indeed reached the late endosome, and most likely escaped it, hence the lower intensity with respect to the early endosome signal.

No substantial evidence of early or late endosomes were observed at 24 hours, which is believed to be most likely due to receptor internalization and degradation, together with the fact that the internalized dendritic pro-drug escaped the late endosome. No evidence of lysosomes was observed at early time points, which is believed to be consistent with the data obtained that suggested late endosomal escape. However, high lysosomal fluorescence signal was recorded at 24 hours. This phenomenon is believed to be most likely due to lysosome-dependent apoptosis, as doxorubicin was known to induce the lysosomal pathway of apoptosis in cancer cells (Sheng, Y, et al. Mol. Pharm. 2015; 12(7): 2217-28).

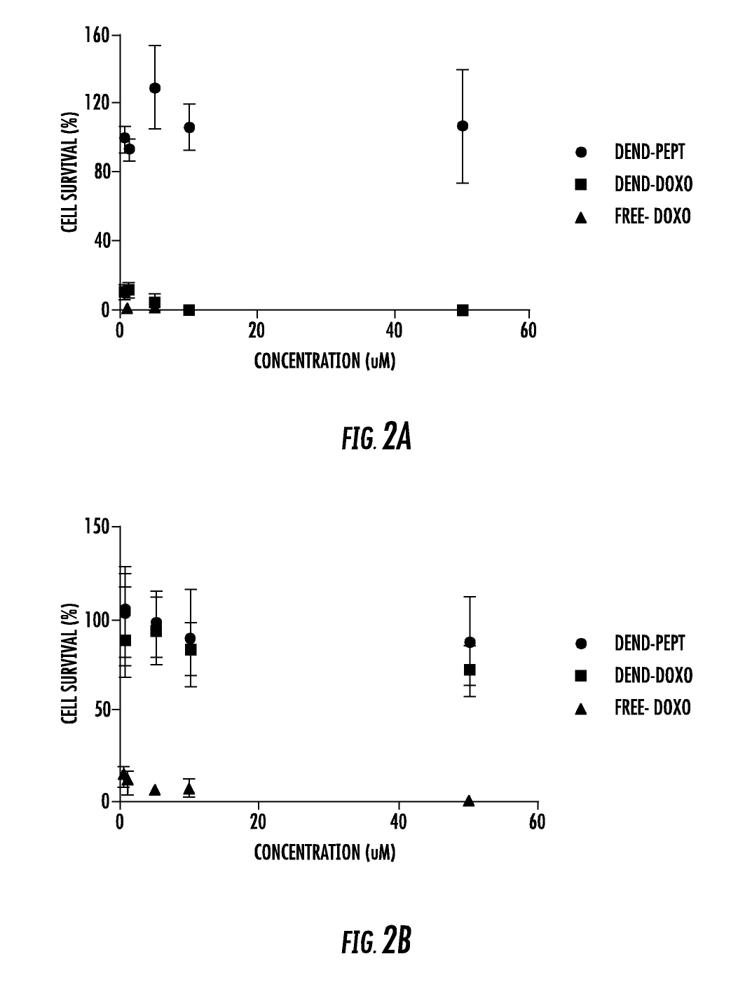

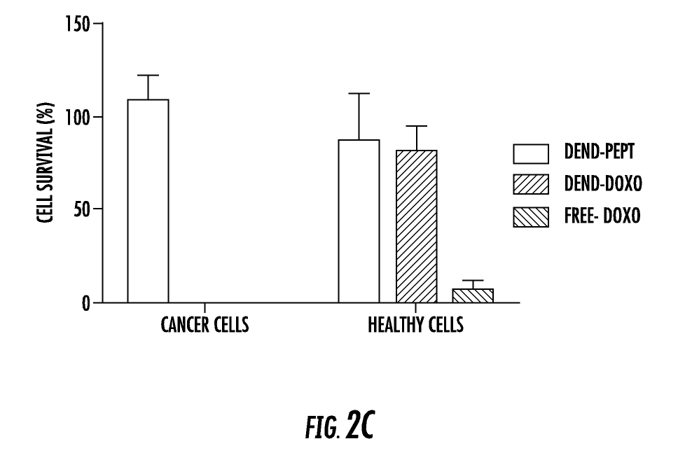

The next question studied was whether conjugated doxorubicin still maintained its cytotoxic effect. Dose-dependent studies showed a cancer-specific cytocidal effect in EGFR-overexpressing cancer cells after 48 hours (cell death between 45% and 96%, as shown at FIG. 2A), while healthy cells exhibited toxicity levels comparable to those of the dendrimer-peptide without the drug, as shown at FIG. 2B.

At a concentration of 10 m, the dendritic pro-drug had a cytocidal effect of over 90% in cancer cells while not affecting healthy cells, as evidenced by a 100% cell survival (FIG. 2C).

The equivalent free doxorubicin concentration caused over 90% cell death in healthy mammary epithelial cells. A decrease in toxicity has been reported when dendrimers were PEGylated, owing to lower cellular uptake (Zhu, S. et al. Pharm. Res. 2010; 27(9): 2030). However, cytotoxic potency of those conjugates was also dramatically reduced with respect to that of free doxorubicin, with IC₅₀ between 27.83 μm and 138.59 μm. By adding the EGF mimicking peptides, the dendritic pro-drug of the examples had an IC₅₀ lower than 0.5 μm.

### Example 6—Dendritic Pro-Drug Hydrogel

Fluorescein-tagged dendritic pro-drug was mixed with a dendrimer solution having a 10% solids content, and then allowed to cure with a solution of fluorescently labeled dextran aldehyde having a 10% solids content. The hydrogel matrix included a 10% solid content PAMAM dendrimer, generation 5, with 75% of the amines of its surface groups oxidized to alcohols (i.e., 32 amines and 96 alcohols), and a 10% solid content dextran aldehyde (10 kDa weight average molecule weight, with 50% oxidation to aldehydes).

Pre-cured 6-mm disks of the doped hydrogel were snap-frozen, cryosectioned, and analyzed by confocal fluorescence microscopy. The dendritic pro-drug was homogeneously distributed in the hydrogel structure, as evidenced by the presence of green fluorescence dye throughout the scaffold in blue.

A three-dimensional rendering of the hydrogel scaffold revealed tortuous and irregular pores of over 100 μm in diameter. The pore size distribution of the scaffold decreased as a function of the tumor selective dendritic pro-drug concentration compared to the empty scaffolds, which suggested that the dendritic nanoparticles were cross-linked into the network.

Release experiments showed dual kinetics-first a burst release of approximately 50% of the total dendritic pro-drug load, followed by a sustained release over three weeks. Free doxorubicin was released in approximately 6 days, owing to the interactions between amine groups in the drug and aldehyde groups in the dextran. When amines in the doxorubicin structure were blocked with aconitic anhydride, the drug was released rapidly through the large pores of the hydrogel in 48 hours. The final concentration of tumor selective dendritic pro-drug in the scaffold (100 or 200 μM) did not affect the degradation kinetics of the scaffold in a statistically significant manner. Nanoparticle percent burst release was approximately half when the final concentration was increased from 100 to 200 μM (burst release of approximately 60̂% and 30%, respectively), corresponding to the same total amount of nanoparticles released (1.9 and 2.1 nanomoles of dendritic pro-drug, respectively). The subsequent sustained release was slightly accelerated (average of 1.53% versus 2.51% per day, or 0.06 versus 0.21 nanomoles per day. This data suggested that the unbound complexes diffused out of the hydrogel scaffold through the big pores, while complexes cross-linked to the scaffold were released as the material degraded over time, as evidenced by the non-linear correlation between the dendritic pro-drug release and the square root of time. The final concentration of dendritic pro-drug in the scaffold (25 or 50 μM) did not appear to affect either the release profile in terms of percent complex released over time, or the degradation rate of the scaffold.

### Example 7—In Vivo Accumulation Following Systemic and Local Delivery

An analysis was conducted of the organ distribution of both the dendritic pro-drugs and free doxorubicin after local and systemic administration. Fluorescently labelled dendritic pro-drugs of Example 5 or free drug were injected systemically and their distributions were assessed after 24 hours. Similar amounts of dendritic pro-drug and free drug accumulated at the tumor site, and no significant accumulation was observed in any other organ (e.g., spleen, heart, lungs, liver, or kidneys) compared to the untreated control. Distribution after local administration by incorporating either the dendritic pro-drug or the free doxorubicin to the hydrogel scaffold was assessed 72-hour post-implantation. Accumulation at the tumor site following local therapy was 5 times higher than that of systemic administration, while no accumulation was observed in other organs compared to the untreated control. No circulating drug or dendritic pro-drug were observed in the blood after local delivery, as evidenced by fluorescence intensities statistically equal to the untreated control. While free doxorubicin concentration in the blood was not statistically different than that of the untreated control after systemic administration, the dendritic pro-drug fluorescence was found to be four times higher than that of free drug, indicating that the dendritic pro-drug had longer circulation time than free drug.

### Example 8—In Vivo Therapeutic Efficacy of Dendritic Conjugates

The therapeutic effect of the doxorubicin-functionalized dendritic pro-drugs of Example 5 crosslinked into a hydrogel scaffold in a murine model of triple negative breast cancer (TNBC) was investigated, and the efficacy of the dendritic pro-drug platform was compared with that of systemic nanoparticle administration and with free drug, administered both locally and systemically.

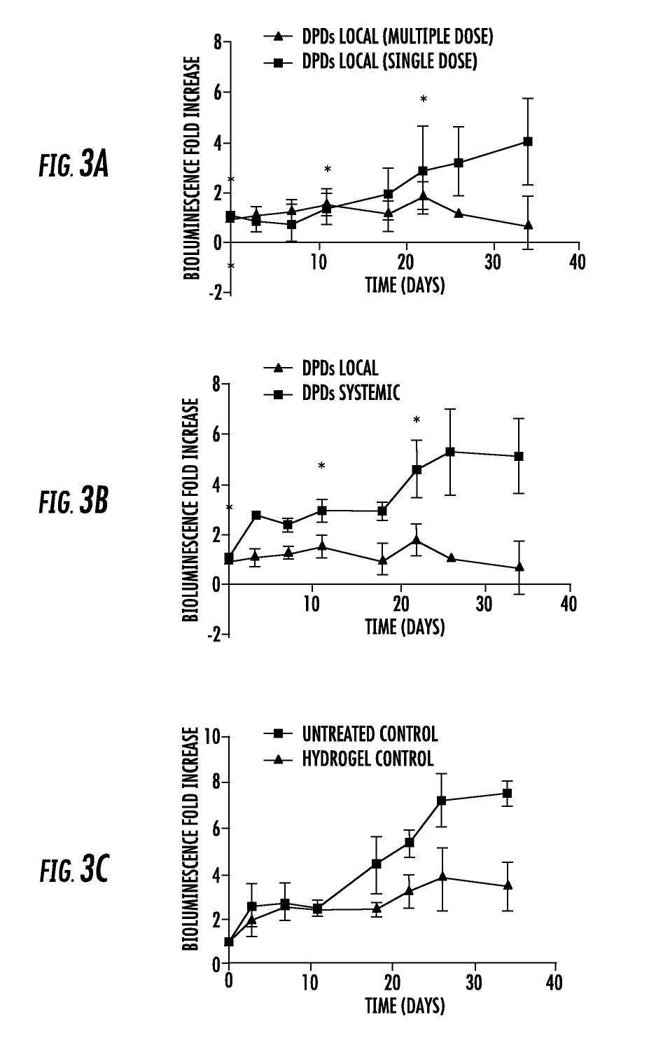

First examined was the effect of release kinetics and dose regimen of dendritic pro-drugs delivered locally on the therapeutic effect. Multi-dose administration consisted of doses of 100 μA of hydrogel doped with 100 μM dendritic pro-drug at days 0, 11, and 22, and was compared to a single dose of 200 μL containing 200 μM dendritic pro-drug at day 0. Efficacy was measured as tumor cell bioluminescence over time, a surrogate for tumor volume. As shown at FIG. 3A, tumor evolution of single and multiple dose regimens of dendritic pro-drugs (DPDs) was similar for the first three weeks, but considerably increased for the single dose group after day 22, which correlated with the complete dendritic pro-drug release from the hydrogel. As shown at FIG. 3B, multiple-dose local and sustained administration of the dendritic pro-drug led to 90% tumor shrinkage compared to the untreated control, while systemic administration only achieved 52% tumor reduction. Control groups included untreated and empty hydrogel scaffold-implanted mice. Empty scaffold implantation achieved 58% tumor reduction compared to untreated control, which suggested that tumor coating with the hydrogel might have mechanically stalled tumor growth (FIG. 3C). When the hydrogel was doped with free drug, which was released faster than the dendritic pro-drug and uptaken non-selectively, the tumor shrinkage was 69%, which was slightly higher but not statistically significantly different than that of the empty hydrogel scaffold. Survival studies showed increased survival for the multi-dose group (between 53 and 63 days) compared to the single dose group (between 30 and 53 days), which correlated with improved therapeutic effect and indicated the importance of the dose regimen, which may be dictated by the release kinetics from the hydrogel. Free doxorubicin administered systemically had the poorest survival rate, even lower than that of the untreated control (between 9 and 20 days versus 30 and 39 days for the free doxorubicin and untreated control), and did not exhibit significant tumor volume reduction compared to the untreated control at its endpoint. The endpoint for mice treated with systemic doxorubicin was determined by poor body condition, excessive weight loss, and reduced mobility of the animals, which took place before tumors reached the pre-established maximum tumor volume of 1 cm³, due to the side effects associated with systemic chemotherapy. On the contrary, mice treated with either dendritic pro-drug (systemically or locally) or with free drug locally did not exhibit the side effects observed with systemic doxorubicin, and were sacrificed when tumors reached the maximum tolerated volume for the experiment.

H&E staining of tumor sections showed a necrotic core, more pronounced in the case of tumors treated with empty hydrogel scaffold compared to the untreated control. Both local and systemic dendritic pro-drug administration led to increased cell death compared to the untreated control. However, tumors treated with local dendritic pro-drugs showed higher cytotoxicity and lower tissue integrity, which correlated with the higher therapeutic effect observed in vivo. Finally, Ki67 stainings showed decreased tumor cell proliferation in the groups treated with dendritic pro-drugs either locally or systemically compared to untreated control or mice treated with the empty scaffold. The levels of Ki67 of tumors treated with empty hydrogels were comparable to those of the untreated controls, which suggested that the therapeutic effect observed in vivo was not due to antiproliferative cytotoxicity caused by the adhesive hydrogel, but instead may have been caused by mechanical constraints around the tumor.

Tumors were rejected from the SCID SHC mice and frozen at −80° C. for 24 h, then embedded with O.C.T and cryosectioned with Leica CM 1850 Cryotome to yield 9 μm tissue slides. Immunostaining against EGFR was performed following standard protocols. EGF receptor (D38B1) XP(R) Rabbit mAb (Cell Signalling) was used as primary antibody and Goat Anti-Rabbit IgG conjugated to Alexa Fluor 647 (Thermo Fisher Scientific) was used as secondary antibody. Cell nuclei were stained with DAPI. Samples were analysed by confocal fluorescence microscopy (Nikon AIR Ultra-Fast Spectral Scanning Confocal Microscope) and images were processed using ImageJ software.

Ki67 immunostainings were performed using the primary antibody Anti-Ki67 (ab15580 Abcam) following a permeabilization step using Triton X 0.5% in PBS for 20 min. Goat Anti-Rabbit IgG conjugated to Alexa Fluor 647 (Thermo Fisher Scientific) was used as secondary antibody and nuclei were stained with DAPI.

Specifically, the mice were randomly sorted into control and experimental groups (n=5 for each group) and injected with either i) 100 μl empty hydrogel, ii) 100 μl hydrogel containing 100 μM TSNPs adjacent to the tumor, iii) 100 μl hydrogel containing 800 μM doxorubicin adjacent to the tumor (equivalent doxorubicin dose to TSNPs), iv) 5 mg/kg doxorubicin intraperitoneally, v) 1 mole/kg TSNPs intraperitoneally (equivalent dose to free doxorubicin) or vi) 200 μl hydrogel containing 200 μM doxorubicin adjacent to the tumor. All the groups except for vi) were dosed again at days 11 and 22. Untreated controls were used to monitor natural disease progression. For local hydrogel implantation, syringes, mixing tips and needles used were autoclaved and for all experimental groups, solutions were sterilized through 0.2 um filters. Tumor progression was assessed by bioluminescence measurements following intraperitoneal luciferin administration using the Xenogen IVIS device. Survival cut-off criteria included tumor ulceration or compassionate euthanasia, due to aggregate tumor burden >1 cm³ in diameter, poor body condition or reduced mobility. All experimental protocols were in compliance with NIH guidelines for animal use.

### Example 9—In Vivo Tumor-Selective Uptake

Whether the selective uptake observed in vitro was maintained in the in vivo setting was tested. Mice were injected with 100 μL of dendrimer: dextran doped with either 100 μM labelled dendritic pro-drug (of Example 5) or the equivalent concentration of doxorubicin adjacent to the tumor. After 72 hours, cryosections of tumor and healthy tissues in intimate contact with the material were analysed by confocal fluorescence microscopy and co-localization studies were performed. Immunostaining against EGFR was performed on the sections to detect tumor cells, and both the dendritic pro-drug and free drug were fluorescently labelled. Nuclei were stained with DAPI. EGFR-overexpressing tumor cells showed dendritic pro-drug uptake, as evidenced by the significantly higher fluorescence intensity of the green channel compared to that of the untreated control (3.43-fold increase in fluorescence of dendritic pro-drug-treated tumors versus untreated control). Co-localization studies showed a high correlation between EGFR-overexpressing cells and dendritic pro-drugs (Pearson's R value 0.63, tM1=0.646, tM2=0.747, Costes P-Value 1.00). Healthy tissues with basal levels of EGFR, as evidenced by the lower fluorescent intensity in the red channel, did not exhibit a statistically significant increase in green fluorescence compared to the untreated control, which indicated that the dendritic pro-drugs were not uptaken by healthy cells. On the contrary, free drug was indiscriminately uptaken by both healthy and cancer cells. Co-localization studies of tumor cell and doxorubicin fluorescence signals showed a correlation between both (Pearson's R value 0.60, tM1=0.794, tM2=0.843, Costes P-Value 1.00). This data corroborated that the selective uptake of dendritic pro-drug observed in vitro is maintained in vivo in a murine model of breast cancer, as opposed to non-selective uptake of free doxorubicin.

1. A composition comprising:
a first dendrimer having at least two branches with one or more surface groups;
a drug conjugated to the first dendrimer; and
a binding peptide conjugated to the first dendrimer;
wherein the binding peptide is configured to bind to a receptor overexpressed by a diseased cell.

2. The composition of claim 1, further comprising a hydrogel, wherein the first dendrimer is dispersed in the hydrogel.

3. The composition of claim 1, wherein the binding peptide is a synthetic peptide.

4. The composition of claim 1, wherein the binding peptide is selected from an EGF mimicking peptide, an FGF-2 mimicking peptide, a VEGF mimicking peptide, a PDGF mimicking peptide, or a combination thereof; and the receptor is EGFR, FGFR-2, VEGFR, PDGFR, or a combination thereof, respectively.

5. The composition of claim 1, further comprising a drug linker, wherein the drug linker is covalently bonded to the first dendrimer and the drug.

6. (canceled)

7. The composition of claim 1, further comprising a binding peptide linker, wherein the binding peptide linker is covalently bonded to the first dendrimer and the binding peptide.

8. The composition of claim 7, wherein the binding peptide linker is a PEGylated amine-to-sulfhydryl crosslinker.

9. The composition of claim 1, wherein an average of about 10 to about 80 molecules of the binding peptide are conjugated to each molecule of the dendrimer.

10. (canceled)

11. The composition of claim 1, wherein an average of about 4 to about 20 molecules of the drug are conjugated to each molecule of the dendrimer.

12. (canceled)

13. The composition of claim 1, wherein at least 10% of the one or more surface groups of the first dendrimer comprise a primary amine or a secondary amine.

14. The composition of claim 1, wherein the diseased cell is a cancer cell.

15. The composition of claim 14, wherein the drug comprises one or more chemotherapeutic agents.

16. (canceled)

17. The composition of claim 1, wherein the first dendrimer comprises a PAMAM G5 dendrimer.

18. A method of delivery of a drug to biological tissue, the method comprising:
providing a first solution comprising a first polymer component comprising a first polymer having one or more aldehydes;
providing a second solution comprising at least one of (i) a second dendrimer comprising at least two branches with one or more surface groups, wherein about 25% to 100% of the surface groups comprise at least one primary or secondary amine, and (ii) a second polymer component comprising a second polymer having one or more amines;
combining the first and second solutions together to produce a hydrogel composite; and
contacting one or more biological tissues with the hydrogel composite,
wherein at least one of the first solution and the second solution comprises the composition of claim 1.

19. The method of claim 18, wherein the composition is substantially evenly dispersed in the first solution, the second solution, or both the first solution and the second solution.

20. The method of claim 18, wherein the concentration of the composition of claim 1 in at least one of the first solution and the second solution is about 0.01% to about 30% by weight of the first solution or the second solution, respectively.

21. The method of claim 18, wherein the concentration of the first polymer component in the first solution is about 0.01 percent to about 30 percent, by weight of the first solution.

22-46. (canceled)

47. A kit for making a hydrogel composite, the kit comprising:
a first part that includes a first solution comprising a first polymer component comprising a first polymer having one or more aldehydes; and
a second part that includes a second solution comprising at least one of (i) a second dendrimer comprising at least two branches with one or more surface groups, wherein about 25% to 100% of the surface groups comprise at least one primary or secondary amine, and (ii) a second polymer component comprising a second polymer having one or more amines,
wherein at least one of the first solution and the second solution comprises the composition of claim 1.

48. The kit of claim 47, further comprising a syringe, wherein the first solution and the second solution are stored in the syringe.

49-50. (canceled)

51. A method for local delivery of a drug to a biological tissue, comprising:
applying to the biological tissue the composition of claim 2; and
permitting the first dendrimer to diffuse from the composition into the biological tissue.

52. A method of treating cancer in a patient, comprising:
administering to the patient an effective amount of the composition of claim 1.

53-54. (canceled)
